(19) United States
(12) Patent Application Publication
(10) Pub. No.: US 2011/0144921 A1
(43) Pub. Date: Jun. 16, 2011

(54) GREENHOUSE GAS EMISSION TRADING SYSTEM, GREENHOUSE GAS EMISSION TRADING APPARATUS, GREENHOUSE GAS EMISSION TRADING METHOD AND PROGRAM

(75) Inventor: Yoshihito ISHIBASHI, Tokyo (JP)

(73) Assignee: Sony Corporation, Tokyo (JP)

(21) Appl. No.: 12/948,238

(22) Filed: Nov. 17, 2010

(30) Foreign Application Priority Data
Dec. 10, 2009 (JP) 2009-280876

(51) Int. Cl. G06F 19/00; G01N 33/00

(52) U.S. Cl. 702/24

(57) ABSTRACT

There is provided a greenhouse gas emission trading system including at least one battery for storing electric power, a measuring unit that measures, after a first amount of electric power has been stored in the battery, when a power supply request is received from the outside, a power amount of electric power having been stored in the battery, and a calculation unit that calculates greenhouse gas emissions for notification to the outside, based on measurement results by the measuring unit and information about emissions of greenhouse gases that were emitted when generating the first amount of electric power.

### BACKGROUND OF THE INVENTION

1. Field of the Invention

The present invention relates to a greenhouse gas emission trading system, a greenhouse gas emission trading apparatus, a greenhouse gas emission trading method and a program.

2. Description of the Related Art

There is an urgent need to limit emissions of greenhouse gases, such as carbon dioxide, methane and nitrous oxide, in order to suppress global warming, and actions to suppress and reduce emissions are taken by countries, local governments and also by companies. Further, emission trading schemes are established that allow apparent emissions to be suppressed and reduced by trading a right to emit greenhouse gases (an emission right).

Furthermore, in order to drastically reduce greenhouse gas emissions, it is necessary to introduce renewable energy such as wind power and sunlight on a large scale. When electric power is generated from a renewable source of energy, the generated electric power may be directly used in a household. Alternately, the generated electric power may be bought or sold from/to an electric power company. However, when a huge amount of electric power is generated in many households, an electric power company could not buy the electric power in a single uniform way in the future. Therefore, it is expected that generated electric power is stored for a time and used later in a household in which the electric power was generated. In such a case, a battery for storing electric power is needed.

Against this background, a carbon dioxide emission reduction system is proposed in which emissions of carbon dioxide as a greenhouse gas are fixed in units of energy and, when supplying energy to a consumer, carbon dioxide emission points are calculated and given (see JP-A-2009-70083, for example).

### SUMMARY OF THE INVENTION

Incidentally, when electric power is stored in a battery, electric power stored in the battery gets gradually lost over time due to self-discharge. On the other hand, emissions of carbon dioxide that was emitted at the generation of the electric power stored in the battery remain unchanged, even if the electric power has been lost from the battery. Accordingly, when the electric power has been lost from the battery, carbon dioxide emissions per unit power of the electric power stored in the battery increase in comparison with those before the electric power gets lost.

However, in carbon dioxide emission trading, a case where electric power is stored as described above is not expected, and there is an issue that it is impossible to accurately perform carbon dioxide emission trading in trading of electric power stored in a battery.

In light of the foregoing, it is desirable to provide a greenhouse gas emission trading system, a greenhouse gas emission trading apparatus, a greenhouse gas emission method and a program, which are novel and improved and which are capable of accurately performing carbon dioxide emission trading in trading of electric power stored in a battery.

According to an embodiment of the present invention, there is provided a greenhouse gas emission trading system including at least one battery for storing electric power, a measuring unit that measures, after a first amount of electric power has been stored in the battery, when a power supply request is received from the outside, a power amount of electric power having been stored in the battery, and a calculation unit that calculates greenhouse gas emissions for notification to the outside, based on measurement results by the measuring unit and information about emissions of greenhouse gases that were emitted when generating the first amount of electric power.

The battery may include a first battery and a second battery. The measuring unit may measure, after a second amount of electric power has been stored in the first battery and a third amount of electric power has been stored in the second battery, when a power supply request is received from the outside, a power amount of electric power having been stored in the first battery and the second battery. The calculation unit may calculate greenhouse gas emissions for notification to the outside, based on measurement results by the measuring unit, information about emissions of greenhouse gases that were emitted when generating the second amount of electric power and information about emissions of greenhouse gases that were emitted when generating the third amount of electric power.

The greenhouse gas emission trading system may further include a transmitting unit that performs notification to the outside of information about the calculated greenhouse gas emissions.

The transmitting unit may perform notification to the outside of information about the calculated greenhouse gas emissions at a predetermined time interval.

The transmitting unit may perform notification to the outside of information about the calculated greenhouse gas emissions, when another power supply request is received from the outside, and after power supply requested from the outside is terminated.

The greenhouse gas emission trading system may further include a recoding unit that records information about emissions of greenhouse gases that were emitted when generating electric power to be stored in the battery.

The recoding unit may further record information about a power amount of electric power to be stored in the battery.

The calculation unit may calculate greenhouse gas emissions for notification to the outside, based on measurement results by the measuring unit, information about emissions of greenhouse gases that were emitted when generating the first amount of electric power, and information about emissions of greenhouse gases that were emitted when generating electric power necessary for maintaining storage of the battery since after the first amount of electric power has been stored in the battery until the power supply request is received.

The calculation unit may calculate greenhouse gas emissions for notification to the outside, based on measurement results by the measuring unit, information about emissions of greenhouse gases that were emitted when generating the second amount of electric power, information about emissions of greenhouse gases that were emitted when generating electric power necessary for maintaining storage of the first battery since after the second amount of electric power has been stored in the first battery until the power supply request is received, information about emissions of greenhouse gases that were emitted when generating the third amount of electric power, and information about emissions of greenhouse gases that were emitted when generating electric power necessary for maintaining storage of the second battery since after the third amount of electric power has been stored in the second battery until the power supply request is received.

According to another embodiment of the present invention, there is provided a greenhouse gas emission trading apparatus including a measuring unit that measures, after a first amount of electric power has been stored in at least one battery for storing electric power, when a power supply request is received from the outside, a power amount of electric power having been stored in the battery, and a calculation unit that calculates greenhouse gas emissions for notification to the outside, based on measurement results by the measuring unit and information about emissions of greenhouse gases that were emitted when generating the first amount of electric power.

According to another embodiment of the present invention, there is provided a greenhouse gas emission trading method, including the steps of measuring, after a first amount of electric power has been stored in at least one battery for storing electric power, when a power supply request is received from the outside, a power amount of the electric power having been stored in the battery, and calculating greenhouse gas emissions for notification to the outside, based on measurement results at the step of measuring and information about emissions of greenhouse gases that were emitted when generating the first amount of electric power.

According to another embodiment of the present invention, there is provided a program for causing a computer to function as a measuring unit that measures, after a first amount of electric power has been stored in at least one battery for storing electric power, when a power supply request is received from the outside, a power amount of electric power having been stored in the battery, and a calculation unit that calculates greenhouse gas emissions for notification to the outside, based on measurement results by the measuring unit and information about emissions of greenhouse gases that were emitted when generating the first amount of electric power.

According to the embodiments of the present invention as described above, it is possible to accurately perform carbon dioxide emission trading in trading of electric power stored in a battery.

### BRIEF DESCRIPTION OF THE DRAWINGS

FIG. 1 is an explanatory diagram illustrating a general configuration of a greenhouse gas emission trading system according to a first embodiment of the present invention;

FIG. 2 is an explanatory diagram illustrating greenhouse gas emission trading processing that is executed by the greenhouse gas emission trading system of FIG. 1;

FIG. 3 is an explanatory diagram illustrating greenhouse gas emission trading processing that is executed by a greenhouse gas emission trading system according to a second embodiment of the present invention;

FIG. 4 is an explanatory diagram illustrating first notification processing of carbon dioxide emissions that is executed by the greenhouse gas emission trading system according to each of the above embodiments; and

FIG. 5 is an explanatory diagram illustrating second notification processing of carbon dioxide emissions that is executed by the greenhouse gas emission trading system according to each of the above embodiments.

### DETAILED DESCRIPTION OF THE EMBODIMENT(S)

Hereinafter, preferred embodiments of the present invention will be described in detail with reference to the appended drawings. Note that, in this specification and the appended drawings, structural elements that have substantially the same function and structure are denoted with the same reference numerals, and repeated explanation of these structural elements is omitted.

Explanation will be made in the following order.

1. Greenhouse gas emission trading system (first embodiment)

2. Greenhouse gas emission trading processing (first embodiment)

3. Greenhouse gas emission trading system (second embodiment)

4. Greenhouse gas emission trading processing (second embodiment)

5. First notification processing of carbon dioxide emissions

6. Second notification processing of carbon dioxide emissions

### [1. Greenhouse Gas Emission Trading System]

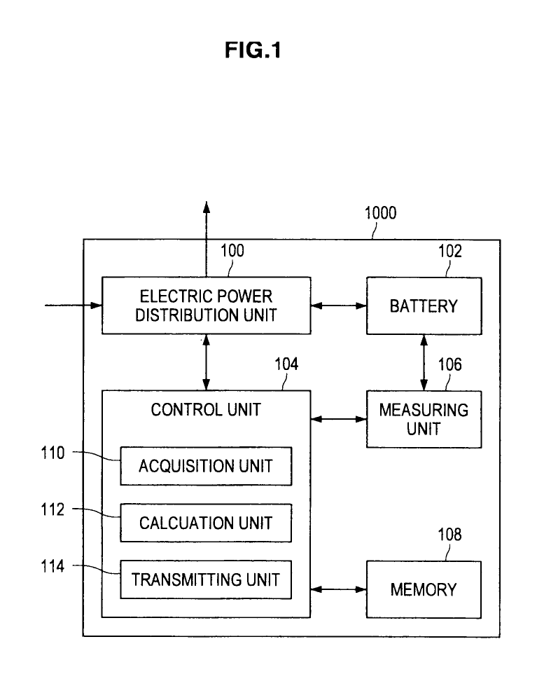

First, a greenhouse gas emission trading system according to a first embodiment of the present invention will be explained. FIG. 1 is an explanatory diagram illustrating a general configuration of the greenhouse gas emission trading system according to the first embodiment of the present invention.

In FIG. 1, the greenhouse gas emission trading system 1000 includes an electric power distribution unit 100, a battery 102, a control unit 104, a measuring unit 106 and a memory 108.

The electric power distribution unit 100 distributes electric power. The battery 102 stores electric power. Electric power supplied from the outside is stored in the battery 102 via the electric power distribution unit 100. Further, the electric power stored in the battery 102 is supplied to the outside via the electric power distribution unit 100. Moreover, in this example, the battery 102 is explained simply as a battery, but what is meant by a battery in a broad sense is every device or system that is capable of storing electric energy in some way and outputting the electric energy on demand. Detailed examples include a currently available storage battery represented by a lithium-ion battery, a nickel hydride battery, a lead storage battery and a NAS battery, a storage battery that would become available in the future, a high-capacitance capacitor represented by a electric double layer capacitor, a transformation system for electric energy and potential energy of water, represented by pumped storage power generation (bringing up water to high altitude by electric energy corresponds to charge of electricity, and guiding water to low altitude and generating electric power by rotating a turbine with water flow corresponds to discharge of electricity), a mutual transformation system between electric power and hydrogen, which is suggested in a hydrogen-recycling-based society (electrolysis of water by electric energy corresponds to charge of electricity, and generating electric power by rotating a turbine while burning hydrogen, or generating electric power using a fuel cell corresponds to discharge of electricity).

The control unit 104 controls the electric power distribution unit 100, the measuring unit 106 and the memory 108. The measuring unit 106 measures a power amount of electric power having been stored in the battery 102. The memory 108 stores information about emissions of carbon dioxide as a greenhouse gas emitted when generating electric power having been stored in the battery 102 and information about a power amount of electric power having been stored in the battery 102.

Further, the control unit 104 includes an acquisition unit 110, a calculation unit 112 and a transmitting unit 114. The acquisition unit 110 acquires measurement results by the measuring unit of a power amount of electric power having been stored in the battery 102. Moreover, the acquisition unit 110 acquires information about carbon dioxide emissions recorded in the memory 108 and information about a power amount. The calculation unit 112 calculates carbon dioxide emissions for notification to the outside, based on measurement results by the measuring unit 106, which were acquired by the acquisition unit 110, and on information about carbon dioxide emissions, acquired by the acquisition unit 110.

The transmitting unit 114 performs notification to the outside of information about the carbon dioxide emissions calculated by the calculation unit 112.

### [2. Greenhouse Gas Emission Trading Processing]

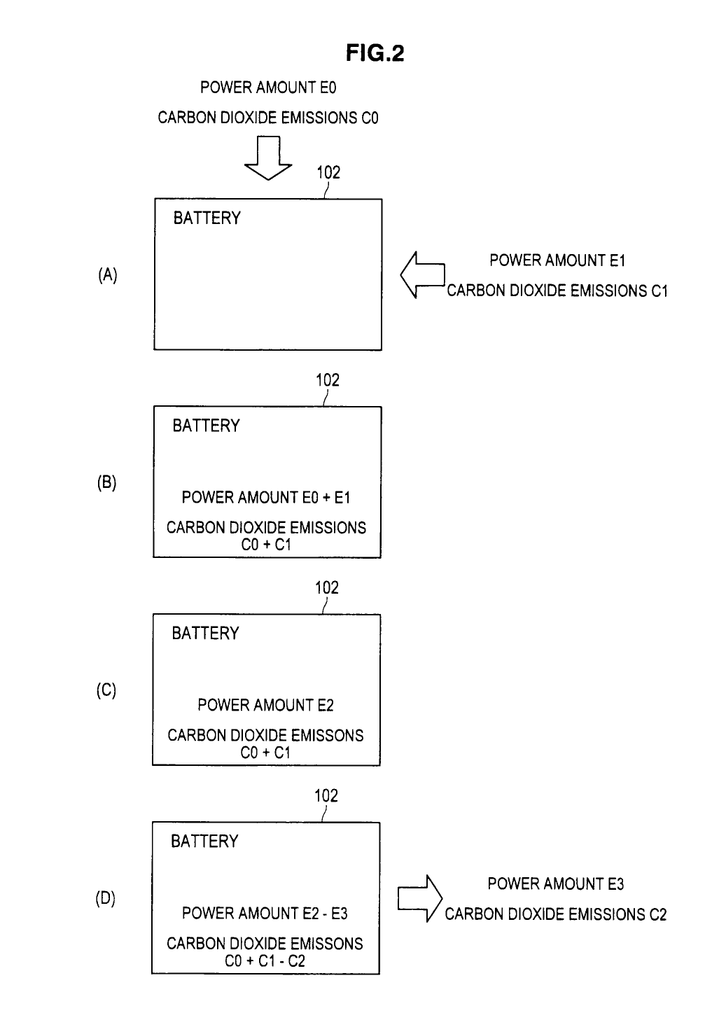

Next, greenhouse gas emission trading processing that is executed by the greenhouse gas emission grading system 1000 of FIG. 1 will be explained. FIG. 2 is an explanatory diagram illustrating greenhouse gas emission trading processing that is executed by the greenhouse gas emission trading system 1000 of FIG. 1.

First, an amount E0 of electric power and information about carbon dioxide emissions C0 as well as an amount E1 of electric power and information about carbon dioxide emissions C1 are supplied via the electric power distribution unit 100 from the outside to the battery 102 ((A) in FIG. 2). Besides, carbon dioxide emissions C0 are emissions of carbon dioxide emitted when generating the power amount E0. Further, carbon dioxide emissions C1 are emissions of carbon dioxide emitted when generating the power amount E1. Here, an amount E0+E1 of electric power, obtained by adding the power amount E0 and the power amount E1, is stored in the battery 102 and carbon dioxide emissions C0+C1, obtained by adding the carbon dioxide emissions C0 and the carbon dioxide emissions C1, are recorded in the memory 108 as carbon dioxide emissions for carbon dioxide emission trading ((B) in FIG. 2).

Thereafter, when the control unit 104 receives a supply request for an amount E3 of electric power from the outside, the control unit 104 causes the measuring unit 106 to measure a power amount of electric power having been stored in the battery 102. Given that, as a result of measurement by the measuring unit 106, the power amount of the electric power having been stored in the battery 102 is a power amount E2 ((C) in FIG. C), relationship between the power amount E0+E1 and the power amount E2 is formed, due to self-discharge in the battery 102, as represented by the following formula 1.

[Expression 1]

E2<E0+E1 Formula 1

Further, the control unit 104 supplies an amount E3 of electric power of the electric power having been stored in the battery 102 via the electric power distribution unit 100 to the outside. Here, in addition to the power supply, the control unit 104 performs notification to the outside of information about carbon dioxide emissions C2 for carbon dioxide emission trading ((D) in FIG. 2). Now, the carbon dioxide emissions C2 are calculated by the following formula 2.

$$\begin{matrix}[ Expression 2 ] & \\ C 2 = \frac{( C 0 + C 1 )}{E 2} \times E 3 & Formula 2\end{matrix}$$

Moreover, after supplying the amount E3 of electric power to the outside, an amount E2−E3 of electric power, obtained by subtracting the power amount E3 from the power amount E2, is stored in the battery 102. And as carbon dioxide emissions for carbon dioxide emission trading, carbon dioxide emissions C0+C1−C2, obtained by subtracting the carbon dioxide emissions C2 from the carbon dioxide emissions C0+C1, are recoded in the memory 108 ((D) in FIG. 2).

According to the greenhouse gas emission trading processing in FIG. 2, when a power supply request is received from the outside, a power amount of electric power having been stored in the battery 102 is measured and carbon dioxide emissions for notification to the outside are calculated based on the measured power amount. Thereby, it is possible to give information about carbon dioxide emissions to the outside, which is calculated in consideration of power loss in the battery 102 due to self-discharge, and therefore it is possible to accurately perform carbon dioxide emission trading in trading of the electric power stored in the battery 102.

Besides, when a NAS battery, for example, is used as the battery 102, the NAS battery is to be kept at high temperature and therefore energy for keeping the NAS battery at the high temperature is needed. Accordingly, when a battery to be kept at high temperature, such as a NAS battery, is used, carbon dioxide emissions related to a power amount of electric power used for keeping the battery at high temperature are to be added to the carbon dioxide emissions that were emitted when generating electric power having been stored in the battery. That is, when a NAS battery, for example, is used as the battery 102, information about carbon dioxide emissions is given to the outside, which were calculated in consideration of the power amount of the electric power used for keeping the battery at high temperature. Thereby, it is possible to accurately perform carbon dioxide emission trading in trading of the electric power stored in the battery 102.

### [3. Greenhouse Gas Emission Trading System]

Next, a greenhouse gas emission trading system according to a second embodiment of the present invention will be explained. The greenhouse gas emission trading system according to the present embodiment differs from that according to the first embodiment only in including, instead of the battery 102, a first battery 116 and a second battery 118. Therefore, explanation of a configuration and effects of the greenhouse gas emission trading system according to the present embodiment will be omitted.

### [4. Greenhouse Gas Emission Trading Processing]

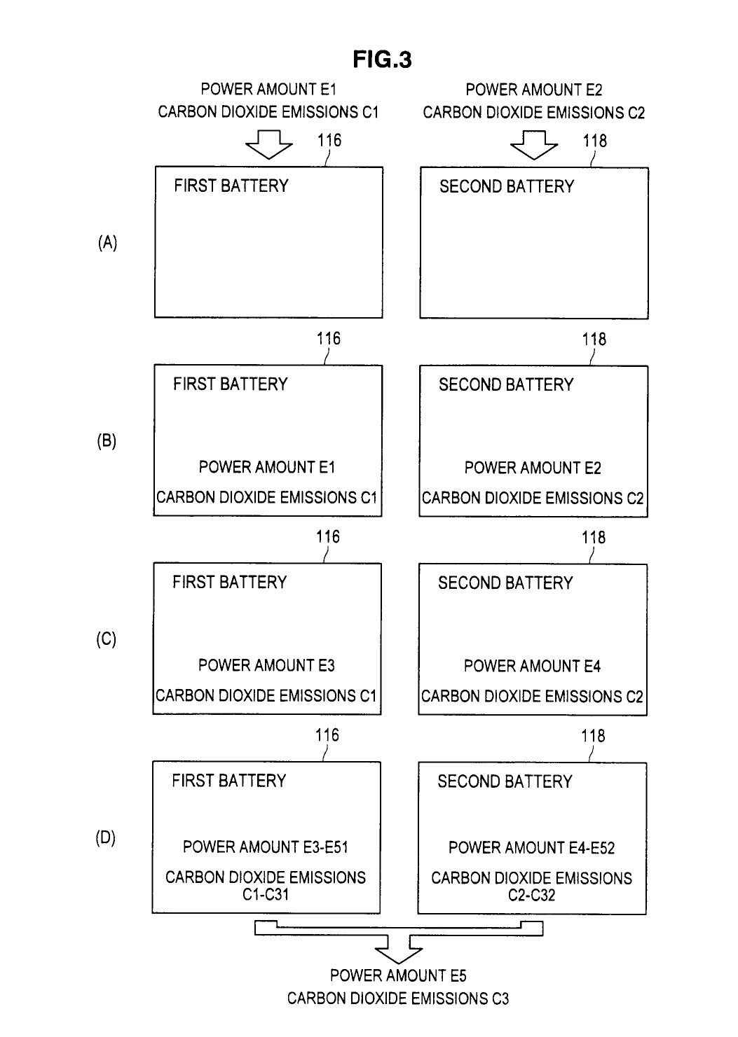

Next, greenhouse gas emission trading processing that is executed by a greenhouse gas emission trading system according to a second embodiment will be explained. FIG. 3 is an explanatory diagram illustrating greenhouse gas emission trading processing that is executed by the greenhouse gas emission trading system according to the present embodiment.

First, an amount E1 of electric power and information about carbon dioxide emissions C1 are supplied via the electric power distribution unit 100 from the outside to a first battery 116. Also, an amount E2 of electric power and information about carbon dioxide emissions C2 are supplied via the electric power distribution unit 100 from the outside to a second battery 118 ((A) in FIG. 3). Besides, carbon dioxide emissions C1 are emissions of carbon dioxide that was emitted when generating the power amount E1. Further, carbon dioxide emissions C2 are emissions of carbon dioxide that was emitted when generating the power amount E2. Here, the amount E1 of electric power is stored in the first battery 116 and the carbon dioxide emissions C1 are recorded in the memory 108 as carbon dioxide emissions for carbon dioxide emission trading. Further, the amount E2 of electric power is stored in the second battery 118 and the carbon dioxide emissions C2 are recorded in the memory 108 as carbon dioxide emissions for carbon dioxide emission trading ((B) in FIG. 3).

Thereafter, when the control unit 104 receives a supply request for an amount E5 of electric power from the outside, the control unit 104 causes the measuring unit 106 to measure a power amount of electric power having been stored in the first battery 116 and a power amount of electric power having been stored in the second battery 118. Given that, as a result of measurement by the measuring unit 106, the power amount of the electric power having been stored in the first battery 116 is a power amount E3 and the power amount of the electric power having been stored in the second battery 118 is a power amount E4 ((C) in FIG. 3), relationship between the power amount E1 and the power amount E3 is formed, due to self-discharge in the first battery 116, as represented by the following formula 3, and relationship between the power amount E2 and the power amount E4 is formed, due to self-discharge in the second battery 118, as represented by the following formula 4.

[Expression 3]

E3<E1 Formula 3

[Expression 4]

E4<E2 Formula 4

Further, the control unit 104 supplies an amount E5 of electric power of the electric power having been stored in the first battery 116 and the second battery 118, via the electric power distribution unit 100 to the outside. Here, in addition to the power supply, the control unit 104 performs notification to the outside of information about carbon dioxide emissions C3 for carbon dioxide emission trading ((D) in FIG. 3). Now, the carbon dioxide emissions C3 are calculated by the following formula 5.

$$\begin{matrix}[ Expression 5 ] & \\ C 3 = \frac{( C 1 + C 2 )}{( E 3 + E 4 )} \times E 5 & Formula 5\end{matrix}$$

Moreover, after supplying the amount E5 of electric power to the outside, an amount of E3−E51 of electric power, obtained by subtracting the power amount E51 from the power amount E3, is stored in the first battery 116. And carbon dioxide emissions C1−C31, obtained by subtracting the carbon dioxide emissions C31 from the carbon dioxide emissions C1, are recoded in the memory 108 as the carbon dioxide emissions for carbon dioxide emission trading. Also, after supplying the amount E5 of electric power to the outside, an amount E4−E52 of electric power, obtained by subtracting the power amount E52 from the power amount E4, is stored in the second battery 118. And carbon dioxide emissions C2−C32, obtained by subtracting the carbon dioxide emissions C32 from the carbon dioxide emissions C2 are recoded in the memory 108 as the carbon dioxide emissions for carbon dioxide emission trading ((D) in FIG. 3). Now, relationship between the power amount E5, the power amount E51 and the power amount E52 is formed as represented by the following formula 6, and relationship between the carbon dioxide emissions C3, the carbon dioxide emissions C31 and the carbon dioxide emissions C32 is formed as represented by the following formula 7.

[Expression 6]

E5=E51+E52 Formula 6

[Expression 7]

C3=C31+C32 Formula 7

According to the greenhouse gas emission trading processing in FIG. 3, when a power supply request is received from the outside, a power amount of electric power having been stored in the first battery 116 and a power amount of electric power having been stored in the second battery 118 are measured and carbon dioxide emissions for notification to the outside are calculated based on the measured power amount. Thereby, it is possible to give information about the carbon dioxide emissions to the outside, which were calculated in consideration of power loss in the first battery 116 and the second battery 118 due to self-discharge, and therefore it is possible to accurately perform carbon dioxide emission trading in trading of the electric power stored in the first battery 116 and the second battery 118. Besides, in the present embodiment, a case where the greenhouse gas emission trading system includes two batteries has been explained, but also in the case of a greenhouse gas emission trading system with two or more batteries, the identical processing can be performed.

### [5. First Notification Processing of Carbon Dioxide Emissions]

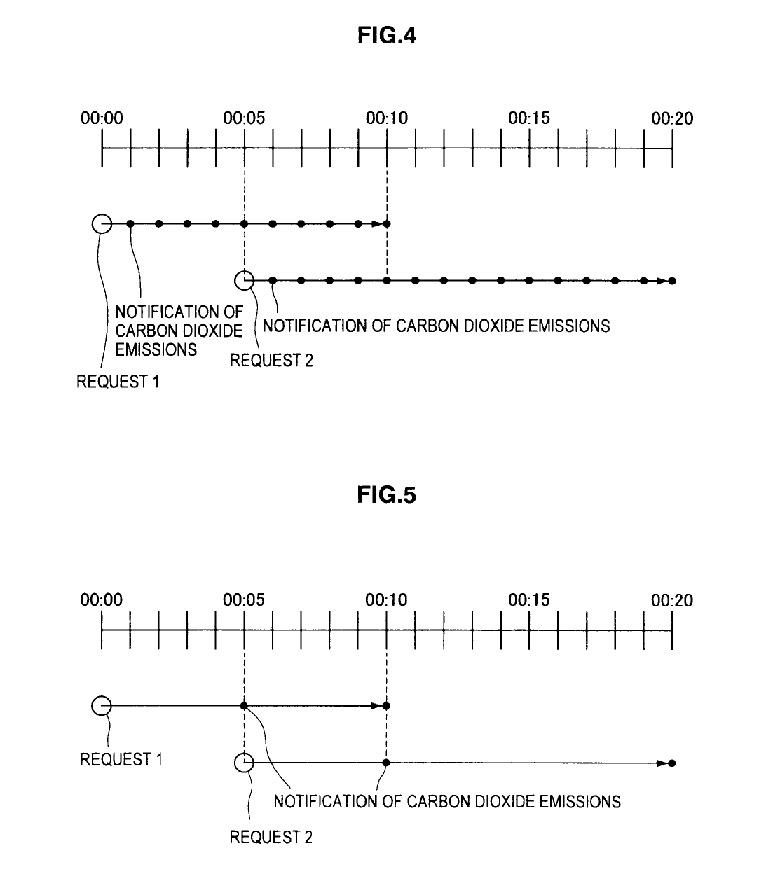

Next, first notification processing of carbon dioxide emissions that is executed by the greenhouse gas emission trading system according to each of the above embodiments will be explained. FIG. 4 is an explanatory diagram illustrating the first notification processing of carbon dioxide emissions that is executed by the greenhouse gas emission trading system according to each of the above embodiments.

In FIG. 4, the control unit 104 of the greenhouse gas emission trading system 1000 receives at time 00:00 a request 1 as a power supply request from the outside. And from time 00:00 until time 00:10, the control unit 104 supplies from the battery 102 to the outside a power amount requested by the request 1. Here, the control unit 104 performs notification to the outside at a predetermined time interval, for example, at one-minute interval, indicating carbon dioxide emissions depending on power amount that has been supplied during the time interval. Besides, in this processing, notification of carbon dioxide emissions is performed at the time indicated by a black circle in FIG. 4.

Further, the control unit 104 receives at time 00:05 a request 2 as a power supply request from the outside. And from time 00:05 until time 00:20, the control unit 104 supplies from the battery 102 to the outside a power amount requested by the request 2. Also here, the control unit 104 performs notification to the outside at a predetermined time interval, for example, at one-minute interval, indicating carbon dioxide emissions depending on power amount that has been supplied during the time interval. Moreover, in a case where a request 2 arrives after 00:04 but before 00:05, power supply processing is to be performed at time 00:05.

Besides, in the present embodiment, the carbon dioxide emissions for notification are calculated by adding up electric power requested by two requests of the request 1 and the request 2. For example, in a case where a power amount of stored electric power at a certain time point is E0 and emissions of carbon dioxide that were emitted when generating the power amount E0 are C0, given that, during time period 00:01, for example, between time 00:06 and time 00:07, electric power E11 is supplied in response to the request 1 and electric power E21 is supplied in response to the request 2, and that carbon dioxide emissions related to the electric power E11, E21 are C11, C12, respectively, relationships are formed as represented by the following Formulae 8, 9.

$$\begin{matrix}[ Expression 8 ] & \\ C 11 = \frac{( E 11 + E 21 )}{E 0} \times \frac{E 11}{( E 11 + E 21 )} \times C 0 & Formula 8 \\ [ Expression 9 ] & \\ C 21 = \frac{( E 11 + E 21 )}{E 0} \times \frac{E 21}{( E 11 + E 21 )} \times C 0 & Formula 9\end{matrix}$$

And at the successive time, a power amount of stored electric power is re-measured and carbon dioxide emissions are re-calculated, so that notification of the carbon dioxide emissions is performed.

According to the first notification processing of carbon dioxide emissions in FIG. 4, notification to the outside is performed at a predetermined time interval, for example, at one-minute interval, indicating carbon dioxide emissions depending on the power amount that has been supplied during the time interval. And thereby, it is possible to accurately perform carbon dioxide emission trading, even if the cessation of power supply is caused.

### [6. Second Notification Processing of Carbon Dioxide Emissions]

Next, second notification processing of carbon dioxide emissions that is executed by the greenhouse gas emission trading system according to each of the above embodiments will be explained. FIG. 5 is an explanatory diagram illustrating the second notification processing of carbon dioxide emissions that is executed by the greenhouse gas emission trading system according to each of the above embodiments.

In FIG. 5, the control unit 104 of the greenhouse gas emission trading system 1000 receives at time 00:00 a request 1 as a power supply request from the outside. Also, the control unit 104 receives at time 00:05 a request 2 as a power supply request from the outside.

From time 00:05 until time 00:10, the control unit 104 supplies from the battery 102 to the outside a power amount requested by the request 1. Here, at the time of the receipt of the request 2, the control unit 104 performs notification to the outside of carbon dioxide emissions depending on power amount that has been supplied until then. And at time 00:10, the control unit 104 performs notification of carbon dioxide emissions depending on power amount that has been supplied from time 00:05 until time 00:10. Besides, in this processing, notification of carbon dioxide emissions is performed at the time indicated by a black circle in FIG. 5.

Further, from time 00:05 until time 00:20, the control unit 104 supplies from the battery 102 to the outside a power amount requested by the request 2. Here, when supply of the power amount requested 1 is terminated at time 00:10, the control unit 104 performs notification to the outside of carbon dioxide emissions depending on power amount that has been supplied until then. And at time 00:20, the control unit 104 performs notification of carbon dioxide emissions depending on power amount that has been supplied from time 00:10 until time 00:20.

According to the second notification processing of carbon dioxide emissions in FIG. 5, when supply of the requested power amount is terminated, notification to the outside of carbon dioxide emissions depending on the power amount that has been supplied until then is performed. Alternately, when another request is received, notification to the outside of carbon dioxide emissions depending on the power amount that has been supplied until then is performed. Alternately, when supply of the power amount requested by such other request is terminated, notification to the outside of carbon dioxide emissions depending on the power amount that has been supplied until then is performed. Thereby, it is possible to reduce the number of transmissions for notification, in contrast to the first notification processing of carbon dioxide emissions in FIG. 4.

Moreover, embodiments of the present invention may be also implemented by providing a system or a device with a recoding medium storing program codes of software that realizes functions of each of the above embodiments and by causing the computer (or CPU, MPU, or the like) of the system or the device to read out and to execute the program codes stored in the recoding medium.

In such a case, the program codes itself, which is read out from the recoding medium, realize functions of each of the above embodiments, and the program codes and the recoding medium storing the program codes constitute the present invention.

Further, as a recoding medium for providing program codes, for example, a floppy disk (registered trademark), a hard disk, a magneto-optical disk, an optical disk such as a CD-ROM, a CD-R, a CD-RW, a DVD-ROM, a DVD-RAM, a DVD-RW, and a DVD+RW, a magnetic tape, a nonvolatile memory card, a ROM or the like may be used. Alternatively, program codes may be downloaded via a network.

Furthermore, functions of each of the above embodiments may be realized not only by executing program codes read out by a computer, but also by causing, based on instructions of such program codes, an operating system (OS) running on the computer to perform a part or whole parts of actual processing.

Moreover, functions of each of the above embodiments may be realized, after program codes read out from a recoding medium are written into a memory which is provided on an extension board inserted into a computer or which is provided in an extension unit connected to a computer, by causing, based on instructions of such program codes, a CPU or the like provided on the extension board or in the extension unit to perform a part or whole parts of actual processing.

It should be understood by those skilled in the art that various modifications, combinations, sub-combinations and alterations may occur depending on design requirements and other factors insofar as they are within the scope of the appended claims or the equivalents thereof.

The present application contains subject matter related to that disclosed in Japanese Priority Patent Application JP 2009-280876 filed in the Japan Patent Office on Dec. 10, 2009, the entire content of which is hereby incorporated by reference.

What is claimed is:

1. A greenhouse gas emission trading system comprising:
at least one battery for storing electric power;
a measuring unit that measures, after a first amount of electric power has been stored in the battery, when a power supply request is received from the outside, a power amount of electric power having been stored in the battery; and
a calculation unit that calculates greenhouse gas emissions for notification to the outside, based on measurement results by the measuring unit and information about emissions of greenhouse gases that were emitted when generating the first amount of electric power.

2. The greenhouse gas emission trading system to claim 1,
wherein the battery includes a first battery and a second battery,
wherein the measuring unit measures, after a second amount of electric power has been stored in the first battery and a third amount of electric power has been stored in the second battery, when a power supply request is received from the outside, a power amount of electric power having been stored in the first battery and the second battery, and
wherein the calculation unit calculates greenhouse gas emissions for notification to the outside, based on measurement results by the measuring unit, information about emissions of greenhouse gases that were emitted when generating the second amount of electric power and information about emissions of greenhouse gases that were emitted when generating the third amount of electric power.

3. The greenhouse gas emission trading system to claim 1, further comprising
a transmitting unit that performs notification to the outside of information about the calculated greenhouse gas emissions.

4. The greenhouse gas emission trading system to claim 3,
wherein the transmitting unit performs notification to the outside of information about the calculated greenhouse gas emissions at a predetermined time interval.

5. The greenhouse gas emission trading system to claim 3,
wherein the transmitting unit performs notification to the outside of information about the calculated greenhouse gas emissions, when another power supply request is received from the outside, and after power supply requested from the outside is terminated.

6. The greenhouse gas emission trading system to claim 1, further comprising
a recoding unit that records information about emissions of greenhouse gases that were emitted when generating electric power to be stored in the battery.

7. The greenhouse gas emission trading system to claim 6,
wherein the recoding unit further records information about a power amount of electric power to be stored in the battery.

8. The greenhouse gas emission trading system to claim 1,
wherein the calculation unit calculates greenhouse gas emissions for notification to the outside, based on measurement results by the measuring unit, information about emissions of greenhouse gases that were emitted when generating the first amount of electric power, and information about emissions of greenhouse gases that were emitted when generating electric power necessary for maintaining storage of the battery since after the first amount of electric power has been stored in the battery until the power supply request is received.

9. The greenhouse gas emission trading system to claim 2,
wherein the calculation unit calculates greenhouse gas emissions for notification to the outside, based on measurement results by the measuring unit, information about emissions of greenhouse gases that were emitted when generating the second amount of electric power, information about emissions of greenhouse gases that were emitted when generating electric power necessary for maintaining storage of the first battery since after the second amount of electric power has been stored in the first battery until the power supply request is received, information about emissions of greenhouse gases that were emitted when generating the third amount of electric power, and information about emissions of greenhouse gases that were emitted when generating electric power necessary for maintaining storage of the second battery since after the third amount of electric power has been stored in the second battery until the power supply request is received.

10. A greenhouse gas emission trading apparatus comprising:
a measuring unit that measures, after a first amount of electric power has been stored in at least one battery for storing electric power, when a power supply request is received from the outside, a power amount of electric power having been stored in the battery; and
a calculation unit that calculates greenhouse gas emissions for notification to the outside, based on measurement results by the measuring unit and information about emissions of greenhouse gases that were emitted when generating the first amount of electric power.

11. A greenhouse gas emission trading method, comprising the steps of:
measuring, after a first amount of electric power has been stored in at least one battery for storing electric power, when a power supply request is received from the outside, a power amount of the electric power having been stored in the battery; and
calculating greenhouse gas emissions for notification to the outside, based on measurement results at the step of measuring and information about emissions of greenhouse gases that were emitted when generating the first amount of electric power.

12. A program for causing a computer to function as
a measuring unit that measures, after a first amount of electric power has been stored in at least one battery for storing electric power, when a power supply request is received from the outside, a power amount of electric power having been stored in the battery; and
a calculation unit that calculates greenhouse gas emissions for notification to the outside, based on measurement results by the measuring unit and information about emissions of greenhouse gases that were emitted when generating the first amount of electric power.
